(19) United States
(12) Patent Application Publication
(10) Pub. No.: US 2025/0114021 A1
(43) Pub. Date: Apr. 10, 2025

(54) INTERACTION-BASED ARTIFICIAL INTELLIGENCE ANALYSIS APPARATUS AND METHOD

(71) Applicant: ELECTRONICS AND TELECOMMUNICATIONS RESEARCH INSTITUTE, Daejeon (KR)

(72) Inventors: Jang-Hee YOO, Daejeon (KR); Cheol-Hwan YOO, Daejeon (KR); Jae-Yoon JANG, Sejong-si (KR); Byung-Ok HAN, Daejeon (KR)

(21) Appl. No.: 18/898,038

(22) Filed: Sep. 26, 2024

(30) Foreign Application Priority Data
Oct. 6, 2023 (KR) 10-2023-0133128
Aug. 20, 2024 (KR) 10-2024-0111014

(51) Int. Cl. A61B 5/16; A61B 5/00; G06T 7/00

(52) U.S. Cl. CPC A61B 5/16; A61B 5/7264; G06T 7/0012; G06T 2207/10016; G06T 2207/30004

(57) ABSTRACT

Disclosed herein are an interaction-based artificial intelligence analysis apparatus and method. The interaction-based artificial intelligence analysis apparatus is configured to output structured video content for each evaluation index so as to elicit an interaction to an examinee at each stimulus time-frame of a preset timeline, collect response data of the examinee for each evaluation index through a camera and a microphone at each response time-frame of the preset timeline, and analyze the response data for each evaluation index using an Artificial Intelligence (AI) analysis module corresponding to the evaluation index.

### CROSS REFERENCE TO RELATED APPLICATIONS

This application claims the benefit of Korean Patent Application Nos. 10-2023-0133128, filed Oct. 6, 2023 and 10-2024-0111014, filed Aug. 20, 2024, which are hereby incorporated by reference in their entireties into this application.

### BACKGROUND OF THE INVENTION

### 1. Technical Field

The present disclosure relates generally to Artificial Intelligence (AI) technology, and more particularly to interaction-based AI analysis technology.

### 2. Description of Related Art

Artificial Intelligence (AI) analysis obtains analysis results by sequentially or in parallel executing various types of AI modules that are capable of detecting responses to audiovisual stimuli, which are previously structured, during limited response times depending on the characteristics of the stimuli based on the stimuli.

Autism Spectrum Disorder (ASD) does not have medical tests, such as blood tests, so it is typically diagnosed by specialists through observation of behavior and development. The observation of behavior and development is primarily performed based on the Diagnostic and Statistical Manual of Mental Disorders, 5th Edition (DSM-5) by the American Psychiatric Association. The DSM-5 presents diagnostic criteria in two areas: the first criterion presents persistent deficits in social interaction and communication, and the second criterion presents restrictive, repetitive, and stereotyped patterns of behavior or interests. The criteria for diagnosing ASD should be based on three key areas of deficit, including the requirement that symptoms need to begin in early childhood, together with the above-described two areas, and should not be focused on one area of deficit.

Currently, as observation and evaluation tools for screening or diagnosing ASD, screening/diagnostic tools that are primarily developed in the United States and Europe are widely used worldwide. Representative tools include the Childhood Autism Rating Scale (CARS), the Modified Checklist for Autism in Toddlers (M-CHAT), the Autism Diagnostic Interview-Revised (ADI-R), and the Autism Diagnostic Observation Schedule (ADOS). Most of these tools involve a procedure of checking off items on a questionnaire based on the observations of parents, primary caregivers, or teachers, or a procedure of checking the responses of an examinee observed during the interaction process between an examiner (or specialist) and an examinee according to the protocols provided by the testing tools.

Early detection and early intervention for ASD are considered highly important in both clinical and research contexts. Although, in the case of ASD, many children are reported to show symptoms between 12 to 24 months of age, and even before 12 months, it is reported that the actual age of diagnosis often takes 3 to 9 years from the time parents first notice concerns about their child's development. This is believed to be due to a lack of information about ASD screening/diagnostic tests and the absence of easily accessible testing tools. Meanwhile, when a psychiatric diagnosis of ASD is necessary, observational assessments using questionnaires are mandatorily conducted. Some research into technology for screening ASD by analyzing perceptual characteristics or visual attention using eye-tracking devices, or by utilizing AI technology to perform emotion recognition or behavior analysis has been conducted. However, because such research is primarily based on a single area of deficit, it can be utilized as a valuable evaluation element, but it is limited in use as a diagnostic tool by non-experts.

Therefore, there is a need to develop an automated method and apparatus which can further objectify and assist the processes by utilizing Information Technology (IT) and Artificial Intelligence (AI) technology, thus realizing more objective screening or diagnostic tests.

Meanwhile, Korean Patent Application Publication No. 10-2023-0119295 entitled “Autism Diagnosis System Using Artificial Intelligence Based on Multi-indicator” discloses an autism diagnostic system using AI based on a multi-indicator (multi-index), which includes a user interface configured to obtain audio data acquired from a subject, video data obtained by capturing the subject, and Childhood Autism Rating Scale (CARS) data of the subject and a server configured to transmit and receive data while communicating with the user interface and to assess the severity of autism spectrum disorder through ensemble analysis with multiple artificial intelligence systems.

### SUMMARY OF THE INVENTION

Accordingly, the present disclosure has been made keeping in mind the above problems occurring in the prior art, and an object of the present disclosure is to comprehensively analyze and assess the responses of an examinee observed during the interaction between an examiner and the examinee, such as in various types of automated Autism Spectrum Disorder (ASD) screening tests and diagnostic support.

Another object of the present disclosure is to efficiently perform AI analysis in limited computing resources by minimizing the driving of an AI analysis module required for analyzing various evaluation indices.

In accordance with an aspect of the present disclosure to accomplish the above objects, there is provided an interaction-based artificial intelligence analysis apparatus, including one or more processors, and memory configured to store at least one program that is executed by the one or more processors, wherein the at least one program is configured to output structured video content for each evaluation index so as to elicit an interaction to an examinee at each stimulus time-frame of a preset timeline, collect response data of the examinee for each evaluation index through a camera and a microphone at each response time-frame of the preset timeline, and analyze the response data for each evaluation index using an Artificial Intelligence (AI) analysis module corresponding to the evaluation index.

The video content may be configured using a scenario for eliciting the interaction based on a characteristic of the examinee and a protocol presented by a test tool.

The timeline may be set to a pair of a stimulus time-frame and a response time-frame for each evaluation index.

The timeline may be formed such that a size of a response collection time-frame corresponding to the stimulus time-frame is individually set for each evaluation index.

The response time-frame may be formed such that a limited time during which response data of the examinee is collected is individually set for each evaluation index.

The at least one program may be configured to output a result of analyzing the response data for each evaluation index.

In accordance with another aspect of the present disclosure to accomplish the above objects, there is provided an interaction-based artificial intelligence analysis method performed by an interaction-based artificial intelligence analysis apparatus, including outputting structured video content for each evaluation index so as to elicit an interaction to an examinee at each stimulus time-frame of a preset timeline, collecting response data of the examinee for each evaluation index through a camera and a microphone at each response time-frame of the preset timeline, and analyzing the response data for each evaluation index using an Artificial Intelligence (AI) analysis module corresponding to the evaluation index.

The video content may be configured using a scenario for eliciting the interaction based on a characteristic of the examinee and a protocol presented by a test tool.

The timeline may be set to a pair of a stimulus time-frame and a response time-frame for each evaluation index.

The timeline may be formed such that a size of a response collection time-frame corresponding to the stimulus time-frame is individually set for each evaluation index.

The response time-frame may be formed such that a limited time during which response data of the examinee is collected is individually set for each evaluation index.

The interaction-based artificial intelligence analysis method may further include outputting a result of analyzing the response data for each evaluation index.

### BRIEF DESCRIPTION OF THE DRAWINGS

The above and other objects, features and advantages of the present disclosure will be more clearly understood from the following detailed description taken in conjunction with the accompanying drawings, in which:

FIG. 1 is a diagram illustrating an interaction-based Artificial Intelligence (AI) analysis apparatus according to an embodiment of the present disclosure;

FIG. 2 is a diagram illustrating an interaction-based Artificial Intelligence (AI) analysis mechanism depending on a structured scenario according to an embodiment of the present disclosure;

FIG. 3 is a diagram illustrating an interaction-based AI analysis processing procedure for each time-frame according to an embodiment of the present disclosure;

FIG. 4 is a block diagram illustrating an interaction-based AI analysis apparatus according to an embodiment of the present disclosure;

FIG. 5 is an operation flowchart illustrating an interaction-based AI analysis method according to an embodiment of the present disclosure; and

FIG. 6 is a diagram illustrating a computer system according to an embodiment of the present disclosure.

### DESCRIPTION OF THE PREFERRED EMBODIMENTS

The present disclosure will be described in detail below with reference to the accompanying drawings. Repeated descriptions and descriptions of known functions and configurations which have been deemed to make the gist of the present disclosure unnecessarily obscure will be omitted below. The embodiments of the present disclosure are intended to fully describe the present disclosure to a person having ordinary knowledge in the art to which the present disclosure pertains. Accordingly, the shapes, sizes, etc. of components in the drawings may be exaggerated to make the description clearer.

In the present specification, it should be understood that terms such as “include” or “have” are merely intended to indicate that features, numbers, steps, operations, components, parts, or combinations thereof are present, and are not intended to exclude the possibility that one or more other features, numbers, steps, operations, components, parts, or combinations thereof will be present or added.

Hereinafter, embodiments of the present disclosure will be described in detail with reference to the attached drawings.

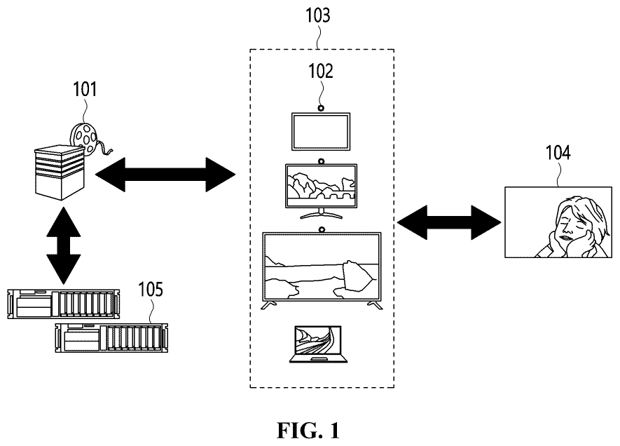

FIG. 1 is a diagram illustrating an interaction-based Artificial Intelligence (AI) analysis apparatus according to an embodiment of the present disclosure.

Referring to FIG. 1, an interaction-based artificial intelligence analysis apparatus 105 according to an embodiment of the present disclosure may play pre-created content 101 via various display devices 103 to elicit interactions depending on a well-refined and organized scenario within a detailed plan.

The interaction-based artificial intelligence analysis apparatus 105 may collect the responses of an examinee 104 who watches the content 101 through a camera 102 and a microphone, store the responses in a computer, and analyze the responses using an Artificial Intelligence (AI) analysis module.

The pre-created content 101 may be played and used via the display devices 103 such as all smart devices equipped with the camera 102.

Each of the display devices 103 may play the content 101 via a computer monitor, a notebook or a television (TV) having a sufficiently large screen and fixed to face forward, thus enabling various types of information of the examinee 104 to be accurately collected.

Further, a server or a computer device corresponding to the interaction-based artificial intelligence analysis apparatus 105 may transmit the pre-created content 101, or may receive and process the collected data.

The interaction-based artificial intelligence analysis apparatus 105 may process and analyze the collected data using computing resources sufficient to effectively execute the AI analysis module.

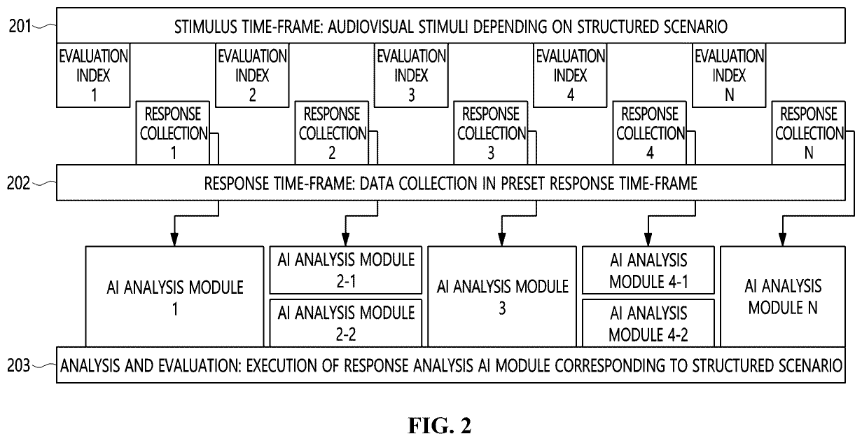

FIG. 2 is a diagram illustrating an interaction-based Artificial Intelligence (AI) analysis mechanism depending on a structured scenario according to an embodiment of the present disclosure.

Referring to FIG. 2, the interaction-based artificial intelligence analysis apparatus 105 may collect examinee response data within a defined stimulus time-frame 201 and a defined response time-frame 202 depending on a structured scenario so as to collect data for Autism Spectrum Disorder (ASD) screening.

The stimulus time-frame 201 may be configured such that video (image) content or the like is pre-created and utilized in the form of audiovisual stimuli depending on the structured scenario so as to suit the purpose of tests. For example, the audiovisual stimuli may be composed of scenarios designed to elicit various social interactions and communications of the examinee, such as the examinee's eye contact, joint attention, name-calling response, imitative behavior, social smiling, social referencing, pointing gesture, and language behavior pattern, based on a protocol presented in an ASD testing tool. For example, the configuration of each scenario may be performed using various methods to naturally elicit responses from the examinee, with respect to speech and playful actions with the examinee and audiovisual stimuli of the video (images) on the screen through a natural interaction in a play manner if possible, under the direction of a host who prompts audiovisual stimuli in the content.

The response time-frame 202 may be an time-frame during which the responses of the examinee to audiovisual stimuli are collected, and may be set to be suitable for content stimuli designed for respective evaluation indices. In the case of behavior such as eye contact, joint attention, name-calling response, social smiling, and social referencing, the response of the examinee may appear during a content time-frame in which responses are elicited. For example, in the case of behavior such as eye contact, there may be a need to set the length of the response time-frame. In the case of behavior such as imitative behavior or social smiling which requires time after stimuli are applied, the length of the response time-frame may be set to a time of about 10 seconds. The length of the response time-frame for behavior such as a pointing gesture to which a relatively fast response may appear may be set to about 5 seconds. Further, the length of the response time-frame for language behavior may be set in consideration of responses that can be predicted differently depending on various types of queries.

Further, the interaction-based artificial intelligence analysis apparatus 105 may configure AI analysis modules corresponding to the evaluation indices of the structured scenario based on the examinee response data that is sequentially collected depending on the structured scenario, and may then analyze the examinee response data and evaluate the results of analysis (203).

Here, the interaction-based artificial intelligence analysis apparatus 105 may include Artificial Intelligence (AI) analysis modules which may effectively analyze responses depending on respective evaluation indices, which are expected based on the collected response data.

For example, the interaction-based artificial intelligence analysis apparatus 105 may sequentially or in parallel execute the AI analysis modules corresponding to gaze tracking for eye contact detection, facial expression recognition for social smiling detection, and head pose estimation for name-calling response detection.

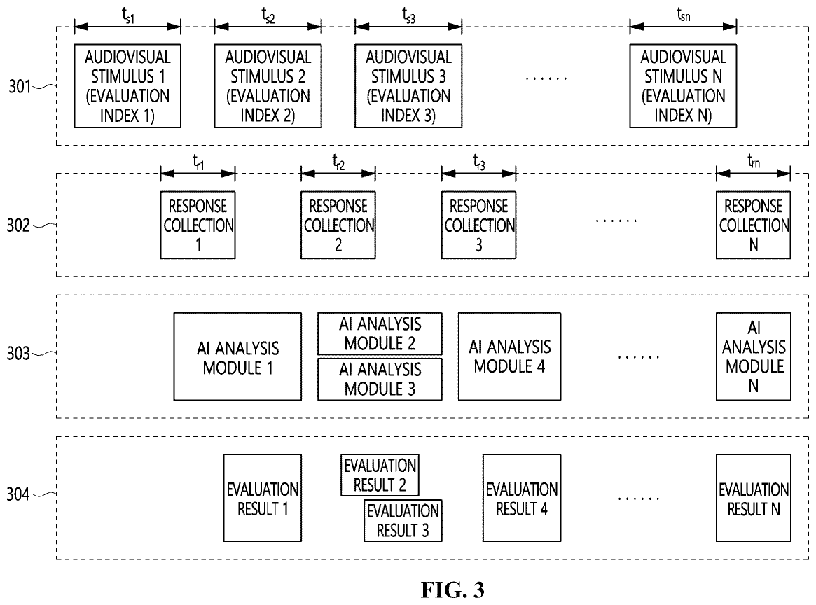

FIG. 3 is a diagram illustrating an interaction-based AI analysis processing procedure for each time-frame according to an embodiment of the present disclosure.

Referring to FIG. 3, it can be seen that the interaction-based AI analysis processing procedure according to an embodiment of the present disclosure is composed of an audiovisual stimulus time-frame 301, a response collection time-frame 302, an AI analysis time-frame 303, and an evaluation time-frame 304.

The audiovisual stimulus time-frame 301 and the response collection time-frame 302 may be set to time-frames on a time axis depending on the structured scenario. Evaluation indices for ASD screening may be structured in the form of the audiovisual stimulus time-frame 301 corresponding to a predefined period in which the time axis is considered, and the response collection time-frame 302 in which data can be collected only within a defined time.

During the AI analysis time-frame 303, the collected data may be analyzed by a mechanism.

During the evaluation time-frame 304, the results of analyzing the data may be output as evaluation results.

The collected data may be divided into time-frames for respective evaluation indices along the time axis, and AI analysis modules corresponding to respective evaluation indices may be used to perform effective AI analysis and produce the results of analysis.

Although the audiovisual stimulus time-frame 301 and the response collection time-frame 302 need to be executed together to collect data in real time, the AI analysis time-frame 303 and the evaluation period 304 based on the results of analysis may be executed offline after collection of data is completed. That is, the AI analysis modules that require considerable computing responses may not be simultaneously executed to perform analysis of collected data, and may be sequentially and structurally executed depending on the corresponding scenario so as to analyze previously structured evaluation indices. Accordingly, computing responses may be more efficiently utilized by performing respective procedures in limited computing resources or by separating a data collection procedure from an analysis procedure.

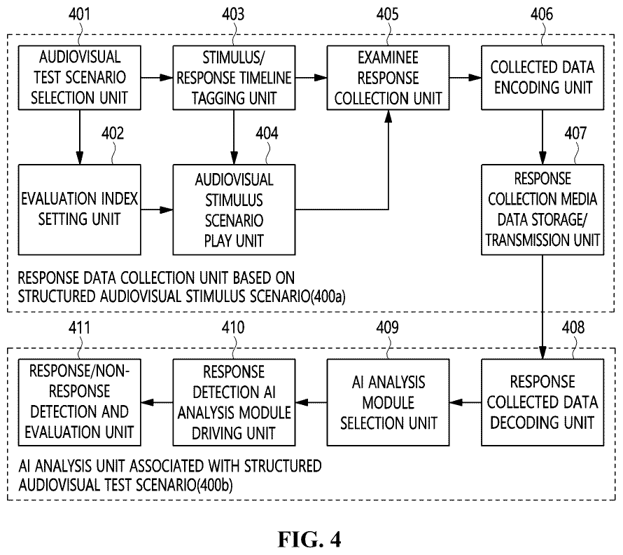

FIG. 4 is a block diagram illustrating an interaction-based Artificial Intelligence (AI) analysis apparatus according to an embodiment of the present disclosure.

Referring to FIG. 4, the interaction-based artificial intelligence analysis apparatus according to the embodiment of the present disclosure may include a response data collection unit 400a based on a structured audiovisual stimulus scenario and an AI analysis unit 400b associated with a structured audiovisual test scenario.

As described above, the response data collection unit 400a based on the structured audiovisual stimulus scenario may be executed to collect data in real time, and the AI analysis unit 400b associated with the structured audiovisual test scenario may be executed to be separated offline after data collection is completed.

The response data collection unit 400a based on the structured audiovisual stimulus scenario may include an audiovisual test scenario selection unit 401, an evaluation index setting unit 402, a stimulus/response timeline tagging unit 403, an audiovisual stimulus scenario play unit 404, an examinee response collection unit 405, a collected data encoding unit 406, and a response collection media data storage/transmission unit 407.

The AI analysis unit 400b associated with the structured audiovisual test scenario may include a response collected data decoding unit 408, an AI analysis module selection unit 409, a response detection AI analysis module driving unit 410, and a response/non-response detection and evaluation unit 411.

The audiovisual test scenario selection unit 401 may select various pre-created digital audiovisual (e.g., video content) scenarios to suit test purposes and personal characteristics in consideration of the gender or age of an examinee, test properties, etc.

Here, the video content may be configured using a scenario for eliciting interactions based on the characteristics of the examinee and a protocol presented by test tools.

Here, the video content may be simplified and structured into a structured audiovisual stimulus time-frame and a response collection time-frame, depending on timelines for respective evaluation indices in the scenario with respect to interactions between an expert based on an ASD screening observation evaluation tool and the examinee.

For example, in order to detect behavior such as eye contact, joint attention, name-calling response, imitative behavior, social smiling, social referencing, pointing gestures, and a language behavior pattern, the video content may be configured using various scenarios designed to observe responses. In detail, in an ASD test tool such as an Autism Diagnostic Observation Schedule (ADOS), there can be used a scenario in which responses are observed through play activities such as free play with specified toys, ball play, responding to name, blocking the examinee from playing with a toy, bubble play, predicting the routine of a subject (e.g., a functional or non-functional toy), predicting a social routine, responding to joint attention, reactive social smiling, doll bath time, functional and symbolic imitation, and snack time.

The evaluation index setting unit 402 may associate evaluation indices for respective time-frames for the structured test scenario in which the examinee is taken into consideration, with the AI analysis modules.

The stimulus/response timeline tagging unit 403 may set the audiovisual stimulus time-frame and the response collection time-frame depending on the timeline based on the selected scenario.

Here, the timeline may be set to a pair of a stimulus time-frame and a response time-frame for each evaluation index.

Here, the timeline may be formed such that the size (or length) of the response collection time-frame corresponding to the stimulus time-frame is individually set for each evaluation index.

Here, the response time-frame may be formed such that a limited time during which the response data of the examinee is collected is individually set for each evaluation index.

The stimulus scenario play unit 404 may output the video content for each evaluation index so as to elicit an interaction to the examinee at each stimulus time-frame of the preset timeline.

The examinee response collection unit 405 may collect response data of the examinee for each of the evaluation indices through a camera and a microphone at each response time-frame of the preset timeline.

The collected data encoding unit 406 may encode data including the timelines and the evaluation indices of the collected response data.

The response collection media data storage/transmission unit 407 may store the encoded response data in a storage device or transmit the encoded response data to another device.

The response collected data decoding unit 408 may decode the encoded response data, together with the timelines and the evaluation indices, so as to perform structured AI analysis on the encoded response data.

The AI analysis module selection unit 409 may select an AI analysis module to suit analysis purpose corresponding to each evaluation index of the decoded response data.

Here, the AI analysis module selection unit 409 may include AI analysis modules that are capable of effectively analyzing expected responses depending on respective evaluation indices based on the collected response data.

The response detection AI analysis module driving unit 410 may sequentially or in parallel drive the AI analysis modules corresponding to the evaluation indices in consideration of limited computing resources.

Here, the response detection AI analysis module driving unit 410 may sequentially or in parallel execute the AI analysis modules corresponding to gaze tracking for eye contact detection, facial expression recognition for social smiling detection, and head pose estimation for name-calling response detection.

The response/non-response detection and evaluation unit 411 may finally evaluate the results of analysis by the AI analysis modules.

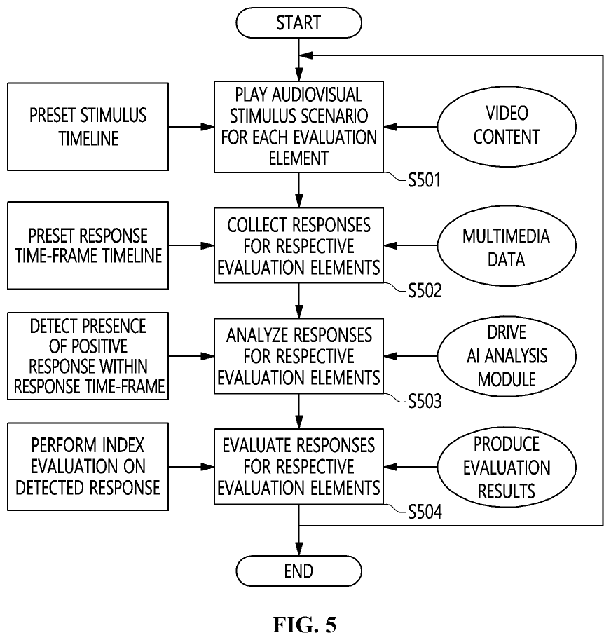

FIG. 5 is an operation flowchart illustrating an interaction-based Artificial Intelligence (AI) analysis method according to an embodiment of the present disclosure.

Referring to FIG. 5, the interaction-based artificial intelligence analysis method according to an embodiment of the present disclosure may play audiovisual stimulus scenarios for respective evaluation elements (indices) at step S501.

That is, at step S501, video content for each evaluation index may be output so as to elicit interactions to an examinee at each stimulus time-frame of a preset timeline.

Here, the video content may be configured using a scenario for eliciting interactions based on the characteristics of the examinee and protocols presented by test tools.

Here, the video content may be simplified and structured into a structured audiovisual stimulus time-frame and a response collection time-frame, depending on timelines for respective evaluation indices in the corresponding scenario with respect to interactions between an expert based on an ASD screening observation evaluation tool and the examinee.

For example, in order to detect behavior such as eye contact, joint attention, name-calling response, imitative behavior, social smiling, social referencing, pointing gestures, and a language behavior pattern, the video content may be configured using various scenarios designed to observe responses. In detail, in an ASD test tool such as an Autism Diagnostic Observation Schedule (ADOS), there can be used a scenario in which responses are observed through play activities such as free play with specified toys, ball play, responding to name, blocking the examinee from playing with a toy, bubble play, predicting the routine of a subject (e.g., a functional or non-functional toy), predicting a social routine, responding to joint attention, reactive social smiling, doll bath time, functional and symbolic imitation, and snack time.

Further, the interaction-based artificial intelligence analysis method according to the embodiment of the present disclosure may collect responses for respective evaluation elements at step S502.

That is, at step S502, response data of the examinee may be collected in the form of multimedia data for each of the evaluation indices through a camera and a microphone at each response time-frame of the preset timeline.

Here, the timeline may be set to a pair of a stimulus time-frame and a response time-frame for each evaluation index.

Here, the timeline may be formed such that the size (or length) of the response collection time-frame corresponding to the stimulus time-frame is individually set for each evaluation index.

Here, the response time-frame may be formed such that a limited time during which the response data of the examinee is collected is individually set for each evaluation index.

Furthermore, the interaction-based artificial intelligence analysis method according to the embodiment of the present disclosure may analyze responses for respective evaluation elements at step S503.

That is, at step S503, the response data may be analyzed using AI analysis modules corresponding to respective evaluation indices.

Here, at step S503, data including the timelines and evaluation indices of the collected response data may be encoded.

Here, at step S503, the encoded response data may be stored in a storage device or transmitted to another device.

Here, at step S503, the encoded response data, together with the timelines and the evaluation indices, may be decoded so as to perform structured AI analysis on the encoded response data.

At step S503, the corresponding AI analysis module may be selected to suit analysis purpose corresponding to each evaluation index of the decoded response data.

Here, at step S503, AI analysis modules corresponding to evaluation indices may be driven sequentially or in parallel in consideration of the limited computing resources.

Here, at step S503, whether positive response of the examinee during each response time-frame appears may be detected using various AI analysis modules.

Here, at step S503, the AI analysis modules corresponding to gaze tracking for eye contact detection, facial expression recognition for social smiling detection, and head pose estimation for name-calling response detection may be executed sequentially or in parallel.

Furthermore, the interaction-based artificial intelligence analysis method according to the embodiment of the present disclosure may evaluate responses for respective evaluation elements at step S504.

That is, at step S504, evaluation results may be produced through index evaluation on responses for respective evaluation elements analyzed or detected by the AI analysis modules.

The structured audiovisual stimuli for ASD screening may be basically configured in the form of content based on the above-described protocol. Although the present disclosure has been described based on ASD screening by way of example, the present disclosure may be utilized for various tests depending on the structured audiovisual test scenarios.

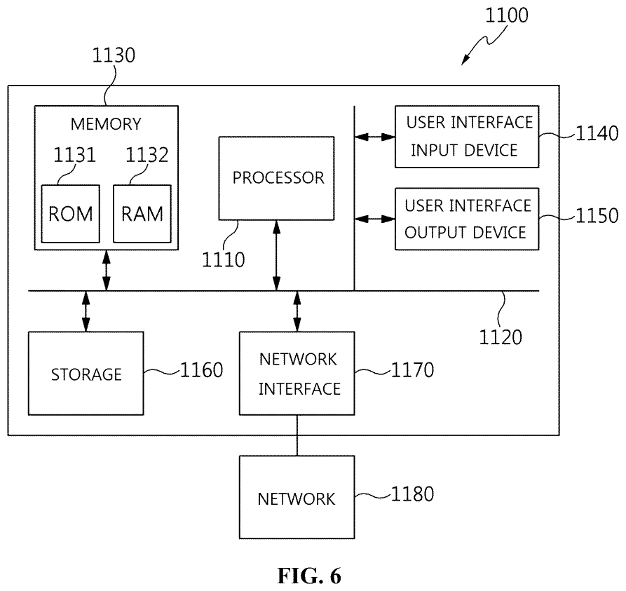

FIG. 6 is a diagram illustrating a computer system according to an embodiment of the present disclosure.

Referring to FIG. 6, the interaction-based artificial intelligence analysis apparatus 105 according to an embodiment of the present disclosure may be implemented in a computer system 1100 such as a computer-readable storage medium. As illustrated in FIG. 6, the computer system 1100 may include one or more processors 1110, memory 1130, a user interface input device 1140, a user interface output device 1150, and storage 1160, which communicate with each other through a bus 1120. The computer system 1100 may further include a network interface 1170 connected to a network 1180. Each processor 1110 may be a Central Processing Unit (CPU) or a semiconductor device for executing processing instructions stored in the memory 1130 or the storage 1160. Each of the memory 1130 and the storage 1160 may be any of various types of volatile or nonvolatile storage media. For example, the memory 1130 may include Read-Only Memory (ROM) 1131 or Random Access Memory (RAM) 1132.

An interaction-based artificial intelligence analysis apparatus according to an embodiment of the present disclosure may include one or more processors 1110 and memory 1130 configured to store at least one program that is executed by the one or more processors 1110, wherein the at least one program is configured to output structured video content for each evaluation index so as to elicit an interaction to an examinee at each stimulus time-frame of a preset timeline, collect response data of the examinee for each evaluation index through a camera and a microphone at each response time-frame of the preset timeline, and analyze the response data for each evaluation index using an Artificial Intelligence (AI) analysis module corresponding to the evaluation index.

Here, the video content may be configured using a scenario for eliciting the interaction based on a characteristic of the examinee and a protocol presented by a test tool.

Here, the timeline may be set to a pair of a stimulus time-frame and a response time-frame for each evaluation index.

Here, the timeline is formed such that a size of a response collection time-frame corresponding to the stimulus time-frame is individually set for each evaluation index.

Here, the response time-frame may be formed such that a limited time during which response data of the examinee is collected is individually set for each evaluation index.

Here, the at least one program may be configured to output a result of analyzing the response data for each evaluation index.

The present disclosure may comprehensively analyze and assess the responses of an examinee observed during the interaction between an examiner and the examinee, such as in various types of automated Autism Spectrum Disorder (ASD) screening tests and diagnostic support.

Further, the present disclosure may efficiently perform AI analysis in limited computing resources by minimizing the driving of an AI analysis module required for analyzing various evaluation indices.

As described above, in the interaction-based AI analysis apparatus and method according to the present disclosure, the configurations and schemes in the above-described embodiments are not limitedly applied, and some or all of the above embodiments can be selectively combined and configured so that various modifications are possible.

What is claimed is:

1. An interaction-based artificial intelligence analysis apparatus, comprising:
one or more processors; and
a memory configured to store at least one program that is executed by the one or more processors,
wherein the at least one program is configured to:
output structured video content for each evaluation index so as to elicit an interaction to an examinee at each stimulus time-frame of a preset timeline,
collect response data of the examinee for each evaluation index through a camera and a microphone at each response time-frame of the preset timeline, and
analyze the response data for each evaluation index using an Artificial Intelligence (AI) analysis module corresponding to the evaluation index.

2. The interaction-based artificial intelligence analysis apparatus of claim 1, wherein the video content is configured using a scenario for eliciting the interaction based on a characteristic of the examinee and a protocol presented by a test tool.

3. The interaction-based artificial intelligence analysis apparatus of claim 1, wherein the timeline is set to a pair of a stimulus time-frame and a response time-frame for each evaluation index.

4. The interaction-based artificial intelligence analysis apparatus of claim 3, wherein the timeline is formed such that a size of a response collection time-frame corresponding to the stimulus time-frame is individually set for each evaluation index.

5. The interaction-based artificial intelligence analysis apparatus of claim 3, wherein the response time-frame is formed such that a limited time during which response data of the examinee is collected is individually set for each evaluation index.

6. The interaction-based artificial intelligence analysis apparatus of claim 1, wherein the at least one program is configured to output a result of analyzing the response data for each evaluation index.

7. An interaction-based artificial intelligence analysis method performed by an interaction-based artificial intelligence analysis apparatus, comprising:
outputting structured video content for each evaluation index so as to elicit an interaction to an examinee at each stimulus time-frame of a preset timeline;
collecting response data of the examinee for each evaluation index through a camera and a microphone at each response time-frame of the preset timeline; and
analyzing the response data for each evaluation index using an Artificial Intelligence (AI) analysis module corresponding to the evaluation index.

8. The interaction-based artificial intelligence analysis method of claim 7, wherein the video content is configured using a scenario for eliciting the interaction based on a characteristic of the examinee and a protocol presented by a test tool.

9. The interaction-based artificial intelligence analysis method of claim 7, wherein the timeline is set to a pair of a stimulus time-frame and a response time-frame for each evaluation index.

10. The interaction-based artificial intelligence analysis method of claim 9, wherein the timeline is formed such that a size of a response collection time-frame corresponding to the stimulus time-frame is individually set for each evaluation index.

11. The interaction-based artificial intelligence analysis method of claim 9, wherein the response time-frame is formed such that a limited time during which response data of the examinee is collected is individually set for each evaluation index.

12. The interaction-based artificial intelligence analysis method of claim 7, further comprising:
outputting a result of analyzing the response data for each evaluation index.
